(19) United States
(12) Patent Application Publication
(10) Pub. No.: US 2007/0123845 A1
(43) Pub. Date: May 31, 2007

(54) METHOD AND DEVICE FOR PROCESSING A WORKPIECE

(76) Inventor: Holger Lubatschowski, Gehrden (DE)

Correspondence Address: ZARLEY LAW FIRM P.L.C., CAPITAL SQUARE, 400 LOCUST, SUITE 200, DES MOINES, IA, 50309-2350, US

(21) Appl. No.: 11/561,600

(22) Filed: Nov. 20, 2006

(30) Foreign Application Priority Data
Nov. 29, 2005 (DE) DE102005056958.7

(51) Int. Cl. A61B 18/18

(52) U.S. Cl. 606/5; 606/10

(57) ABSTRACT

The present invention concerns a method for processing an at least partially fluid-absorbent and at least in one spectral range transparent workpiece. The method is characterized by irradiating the workpiece with pulsed and focused laser radiation, wherein the spectrum of the laser radiation comprises at least one wavelength in the transparent spectral range of the workpiece, and the focus of the laser radiation is positioned on or within the workpiece. Before and/or after the irradiation, a fluid photo-sensitizer is applied onto the workpiece, this photo-sensitizer having an absorption peak at or near half a wavelength of the laser radiation. The invention also concerns a device for performing such a method.

### BACKGROUND OF THE INVENTION

The present invention is directed to a method for processing a workpiece, as well as to a device usable for this purpose.

A conventional method and a corresponding device are known from WO 2005/074848 A1 and from the article “Riboflavin/Ultraviolet-A-Induced Collagen Cross Linking for the Treatment of Keratoconus” G. Wollensak et al., American Journal of Ophthalmology, May 2003, page 620. The method and device are used therein for treating keratoconus. Keratoconus is a disease of the eye, under which the cornea of the eye becomes thinner and is reduced in rigidity and stability. Under the influence of the internal pressure of the eye, this weakening of the cornea leads to the same bulging outwards, which in turn leads to the eye becoming ametropic. Since keratoconus is a progressing disease, there is a considerable risk of the ametropia becoming more severe if the disease is not treated.

In the above mentioned documents, a method and a device for treating keratoconus are suggested. They are both based on the consideration that a cross linking of the collagen fibers in the cornea may increase the rigidity of the cornea, such that the cornea may better resist the internal pressure of the eye. For this purpose, a photo-sensitizer is applied onto the eye, in particular riboflavin or a riboflavin-solution. In general, a photo-sensitizer is a substance which, under the influence of photons, is able to chemically react with the material absorbing the photo-sensitizer or to produce a chemical altering of this material, for example by cross linking molecules and/or increasing the rigidity of the material. After the UV (ultraviolet) sensitive riboflavin has been absorbed by the eye, the eye is exposed to an irradiation with UV radiation. Either a large UV lamp or an array of smaller light sources is used as the UV light source. In each case, it is the express aim to homogeneously irradiate the frontal surface of the complete cornea, in order to homogeneously solidify the cornea. Under the influence of the ultraviolet radiation, the photo-sensitizer induces a cross linking of the collagen fibers, thereby increasing the biomechanical rigidity of the cornea, such that the cornea is likely to deform less under the influence of the internal pressure of the eye.

This conventional method is not without risk for the patient. In the above mentioned article “Riboflavin/Ultraviolet-A-Induced Collagen Cross Linking for the Treatment of Keratoconus” of G. Wollensak et al, it is pointed out that the eye may be damaged if too much UV radiation reaches the lens of the eye or the retina.

However, in this conventional method, the complete thickness of the cornea us available for absorbing the UV radiation, since the UV radiation impinges from the frontal surface of the cornea. It may therefore be expected (and it is desired) that the UV light is considerably attenuated before impinging on the internal or rear portions of the eye.

One of the disadvantages of the conventional method is the cell damaging, cytotoxic effect of the UV radiation. Also, it is unpleasant for the patient that the UV radiation is intended to last for half an hour per eye.

Another method, which probably also requires a long duration of the treatment, is disclosed in US 2005/0149006A1. Contrary to WO 2005/074848 A1, the material of the cornea is now “softened” by heating the cornea and by ablating and destroying material in the bulk of the cornea by means of focused laser light. It is very likely that the destroyed portions will later impair vision of the patient. After softening the cornea, the cornea is brought into a desired shape, provided with a photosensitizer and—as known from WO 2005/074848 A1—irradiated homogeneously by a UV lamp on a large area on its frontal surface, in order to reverse the preceding softening. The destroyed portions remain

Further, JP 2004113322 A does not disclose a therapeutical, but a diagnostical apparatus, using 2-photon-excitation for obtaining images from the rear portion (the “fundus”) of the eye.

The object of the present invention is to improve the conventional method in such a way that it may be performed faster and safer. Further, a device for performing the method shall also be provided.

This object is solved by a method with the features of claim 1, and by a device with the features of claim 21, respectively. The sub claims are directed to advantageous improvements of the invention.

### SUMMARY OF THE INVENTION

According to the present invention, a fluid photo-sensitizer is applied onto the transparent, at least partially fluid-absorbent workpiece. The photo-sensitizer is absorbed by the workpiece, before the workpiece is irradiated with pulsed and focused laser radiation. In this context, “at least partially fluid-absorbent” means that the workpiece may comprise areas which absorb only little or no fluid. The laser radiation and the photosensitizer are adapted to each other in such a way that a wavelength of the laser is approximately at twice the wavelength of an absorption peak of the photo-sensitizer, e.g. with a deviation of +/−10% of twice the absorption peak. A variation of the invention is also possible in which the wavelength of the laser is within corresponding deviations from 3-times, 4-times, or n-times the wavelength of an absorption peak of the photo-sensitizer.

In the invention, the pulsation of the laser radiation in connection with a spatial focusing achieves already at very low and non-damaging doses or average powers of the laser such a high intensity that the probability for 2-, 3-, or multi-photon processes is considerably increased at the focus. By these processes, the photo-sensitizer is activated, such that it induces an altering of the material properties of the workpiece, in particular an increase in hardness or rigidity.

One advantage of the present invention is that—in comparison with the absorption peak of the photosensitizer—light with a rather long wavelength is irradiated onto the workpiece. Light with such a long wavelength alone does not induce any alteration of the workpiece, in particular no damaging. The alteration of the material is confined entirely to the focus of the laser. Outside the focal volume, the intensity is reduced rapidly, such that an activation of the photo-sensitizer does not or only hardly occur. Another advantage of the present invention is that by choosing the parameters of the laser radiation and the focusing, the size of the focal volume is adaptable and variable. In this way, the volume within which the material properties of the workpiece are altered may be varied.

The method of the present invention is not only applicable on the cornea, as described in the prior art. Rather, it may be applied onto all kinds of transparent, fluid-absorbent workpieces. For example, it may be applied on samples of biological material, on extracted or artificially generated samples of biological or organic material, on plants or also onto suitable artificial or plastic materials. For example, plants or biological material may be selectively hardened in certain areas in order to examine them more easily or to improve their growth.

The present invention is directly opposed to the criteria used in the prior art, in particular the conventional demand for a homogenous irradiation of the cornea and for an irradiation only on the surface of the workpiece. Due to the transparency of the workpiece for the laser radiation used, the laser light may not only be effective on the surface, but also on any other desired position or locus within the workpiece. This circumstance renders the method of the present invention very flexible, since the position of the focus, and, hence, the locus of the material processing may be adapted to the specific requirements of each workpiece. In contrast to a large area, homogeneous irradiation, the irradiation in the present invention is initially limited to a spatially very confined position. In addition, the irradiation of the material is very efficient, since due to the transparency of the workpiece and due to the absence of absorption of the laser light in front of the focus, the radiation may be brought to the focus and to the locus of processing generally without attenuation. Due to this increase of efficiency, the processing may also be performed faster.

It is also possible to employ the method for not totally transparent workpieces. However, the workpiece may then best be processed efficiently at or near its surface.

Further advantages may be achieved by the inventive method, if the position of the focus of the laser radiation on or within the workpiece is varied during the processing. For this purpose, either the workpiece is moved relative to the focus and/or the position of the focus is moved relative to the workpiece. It is advantageous if this relative motion is performed between two consecutive laser pulses. Due to this variable position of the focus, the processing or the hardening of the workpiece are not limited to the size of a focal volume, but any desired regions of the workpiece may be processed.

For example, the focus of the laser beam may be guided in such a way that in total it scans one-, two-, and/or three dimensional irradiation zones on or within the workpiece. These irradiation zones at which the workpiece for example is hardened, may be located in any desired way within the workpiece. In contrast to the conventional method, the method of the present invention is selective in processing the workpiece. Hence, for example sensitive areas of the workpiece or such areas, in which due to the properties of the material no hardening may be achieved, may be excluded from irradiation, thereby making the method even more efficient. An irradiation zone may be constituted by a plurality of separately irradiated, adjacent focal volumes, thus, it is not homogeneous on a microscopic scale.

In a variation of the method, the focus of the laser radiation is guided in such a way that it scans line-shaped irradiation zones, and at least two of these line-shaped irradiation zones mutually intersect. In this way, a web with any desired shape is formed on or within the workpiece, the workpiece being hardened along this web. Such a web-shape of the irradiation zones may sometimes lead to an even greater increase in rigidity than a smooth, homogeneous irradiation.

The method becomes even faster and more efficient if the laser radiation is focused simultaneously onto more than one locus. In this aspect of the invention, it is desirable that comparable conditions are present at all foci, in particular, comparable intensities.

Preferably, the laser radiation comprises radiation from the red or infrared region, for example with wavelengths from 600 nm to 1200 nm. This spectral range is particularly advantageous with workpieces of biological material, since this radiation does not have any cell damaging effects.

Preferable, a short pulse or an ultra short pulse laser is used for the inventive method. Even at very low pulse energies—together with correspondingly low collateral damaging effects—such lasers may achieve high intensities at the focus.

For example, the laser pulses may be nanosecond pulses (with a duration of one nanosecond to one microsecond), picosecond pulses (with a duration of one picosecond to one nanosecond), femtosecond pulses (with a duration of one femtosecond to one picosecond), or attosecond pulses (with a duration of up to one femtosecond). The best compromise between sufficiently short pulses and a laser system that is not too demanding in price and maintenance should be a femtosecond or picosecond laser system, for example a titanium:sapphire- or a fiber-femtosecond laser.

If the energy of one laser pulse is at or between one pJ (picojoule) and several 100 nJ, good to excellent processing results may be obtained, depending on the workpiece.

In principle, the method may be performed at any desired pulse repetition rate of the laser. However, in view of the duration of the processing, it is advantageous to use repetition rates of the laser between several 100 Hz and several 100 MHz. Ideal repetition rates are at several MHz, such that the position of the focus within the workpiece may still be varied between two pulses, if desired.

Depending on the choice of workpiece and photo-sensitizer, the laser radiation is preferably applied in such a way that on one locus on or within the workpiece, an energy density of 0.1 kJ/cm² up to several 100 kJ/cm² is deposited, more particularly between 10 kJ/cm² and 150 or 200 kJ/cm². This energy density does not have to be deposited there by one single laser pulse only. Rather, it is also possible to direct several consecutive laser pulses onto a single locus until depositing the desired energy density.

If it is intended to process e.g. biological material, a photo-sensitizer with an absorption peak in the ultraviolet range may be used, in particular riboflavin (also denominated lactoflavin or vitamin B2). Due to its absorption peak in the ultraviolet range, riboflavin may be activated in the method of the present invention by focused red or infrared radiation, which does not influence or damage the biological material outside the focal volume.

In certain applications, it may be advantageous to maintain the workpiece in a predetermined shape by means of a shaping member during the processing. If the workpiece is hardened by the irradiation, it may subsequently remain in the predetermined shape also without the shaping member.

When using a shaping member for stabilizing the workpiece in a predetermined shape, it is particularly advantageous if the shaping member is transparent for the laser radiation and the radiation is applied through the shaping member onto the workpiece. The shaping member may then be comparable to a contact lens that is set onto the workpiece in order to maintain same in the desired shape during the irradiation.

In addition to the processing method, the invention also provides a method of calculation the positions of a plurality of foci in view of performing a method described supra, but before actually processing the workpiece. This calculation considers the influence of the properties of the material, the laser and the photo-sensitizer, and it may be adaptable with respect of achieving a predetermined stabilizing effect on the workpiece.

The present invention is also reflected by a calculation of the positions of a plurality of foci in preparation for performing a method as described above. For the purpose of this calculation, at first the actual shape of the workpiece is measured or analysed, and a desired shape, deviating from the actual shape, is established. The calculation accounts for the material properties of the workpiece ond of the photo-sensitizer, and may also consider the change of volume of the workpiece due to absorption of the photo-sensitizer. Eventually, the optimum laser parameters (e.g. average power) as well as the duration of treatment and the irradiation pattern (i.e. the position and sequence of the foci) will be calculated.

Further, the invention also provides a device for processing a workpiece. This device comprises a short pulse or ultra short pulse laser, a beam guiding system comprising a focusing element, and means for applying a photo-sensitizer onto the workpiece. As already explained, the laser may be a nano-, pico-, femto-, or attosecond pulse laser, wherein femto- and picosecond pulse lasers are preferred. The beam guiding system should be adapted in such a way that the focused of the laser radiation is positioned on or within the workpiece. The means for applying the photo-sensitizer may comprise one or several apertures, via which the photo-sensitizer may be applied onto the workpiece. It is also possible that the means may comprise a pump, for example an electrically operated pump.

The device of the present invention may further comprise positioning means for positioning the workpiece, for example a corresponding holder, in particular also with suitable fixing elements for a secure mounting of the workpiece. This is advantageous in order to be able to position the focus precisely onto the desired loci in the material. If the positioning means is movable, it may also be moved for a relative motion between workpiece and focus.

It is advantageous if the beam guiding means comprises a controllable focusing optics in order to control the position of the focus of the laser radiation, in particular the depth of the focus within the workpiece. By means of such an optics, the focus may be moved faster within the workpiece than with a movement of the workpiece. For example, the depth of the focus within the workpiece may be controlled by a mechanism for moving a focal lens. A spacer may also be provided for controlling the depth, this spacer determining the distance between the focusing optics and the workpiece, as well as distance sensors for controlling this distance. In view of a lateral deflection of the focus relative to the optical axis, the beam guiding system may comprise a scanner system, which conventionally comprises two pivotable mirrors with mutually orthogonal pivoting axes.

In a variation of the device of the present invention, the beam guiding system has a focusing optics that simultaneously generates more than one focus. For example a lens array may be used for this purpose, each lens generating its own focus. The foci may e.g. be located on a planar or curved surface. This variation of the invention has the advantage of simultaneously processing several loci within the workpiece, such that the processing becomes faster. It is conceivable to control a multi-focal optics in such a way that the foci are commonly adjustable, in order to scan the desired irradiation zone within the workpiece.

Depending on the degree of focusing and the chosen repetition rate, the average laser power may preferably be 0.5 to 1000 mW. In order to be able to process very fine structures on or within the workpiece, the focusing should occur at a rather large numerical aperture (NA). Depending on the working distance and on the area to be treated, the numerical aperture could be 0.3 to 1.4.

In order to always have a sufficient supply of photo-sensitizer, it is convenient if the device comprises a reservoir of the photo-sensitizer, which might also be connected to the means for applying the photo-sensitizer.

In addition, it is convenient if the means for applying the photo-sensitizer comprises a metering means in order to control the dosage of the photo-sensitizer. For example, the metering means could make sure that additional portions of photo-sensitizer are applied onto the workpiece in regular temporal intervals.

An atomization and a corresponding further increase of efficiency may be achieved by the device comprising a control for controlling the laser, the elements of the beam guiding system, and/or the means for applying the photo-sensitizer. By means of the control, which may comprises a programmable processor, the components of the device may be adjusted and adapted to each other in an optimal way; and the device may even be automated.

The invention further comprises the use of a short pulse or ultra short pulse laser for producing a device for treating keratoconus. This device could also be used for the treatment of a cornea, i.e the “workpiece” would then be the cornea of the eye of a patient. The laser beam could be guided onto the cornea via a slit lamp arrangement. In this case, it would be convenient if the device comprises a means for stabilizing the position of the eye, for example a suction ring to be applied onto the eye. A couch could further improve the comfort of the patient.

Further, the invention comprises the use of a short pulse or an ultra short pulse laser for producing a device for treating previously determined ametropic characteristics of the eye. Again, the “workpiece” would be the cornea of a patient, and again the laser beam could be guided onto the cornea via an apparatus similar to a slit lamp. Such a device would preferable be used in connection with a shaping member for the eye, for example a contact lens, in order to maintain the eye in an emmetropic shape, in which the cornea could then be hardened.

### BRIEF DESCRIPTION OF THE DRAWINGS

In the following, a preferred embodiment corresponding to the best mode of the invention is described in more detail with respect to the enclosed drawings. In particular,

FIG. 1 shows a schematic view of a device according to the best mode of the present invention,

FIG. 2 shows a schematic view of a part of another embodiment of the device, and

FIG. 3 shows a perspective view of a workpiece after processing with the method of the present invention.

### DETAILED DESCRIPTION OF THE PREFERRED EMBODIMENTS

Like parts are denominated by like reference numerals throughout all figures.

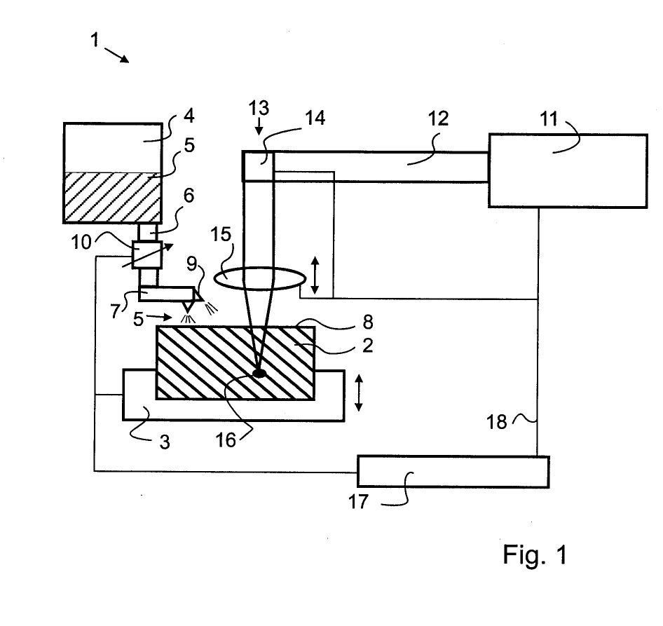

FIG. 1 shows a schematic view of a device 1 according to the present invention for processing an at least partially fluid-absorbent workpiece 2. The workpiece 2 is positioned in a positioning means 3, which here is a holder. The workpiece may also be fixed by fixing means (not shown). The positioning means 3 is movable in different spatial directions. One of these possible directions is indicated in the drawing.

The device 1 comprises a reservoir for accommodating and storing a photo-sensitizer 5, for example a riboflavin solution (in the following: riboflavin). The photo-sensitizer 5 may flow from the reservoir 4 via a line 6 to a means 7 for applying the photo-sensitizer. The applying means 7 is adapted to apply the photo-sensitizer 5 in a suitable way onto a surface 8 of the workpiece 2. For this purpose, the applying means 7 is provided with several applying apertures 9, from which the photo-sensitizer 5 may exit and reach the surface 8 of the workpiece 2 in the form of drops, in the form of a mist, or in the form of a film. In the device 1 of FIG. 1, the applying apertures 9 are formed as spray nozzles.

A metering means 10 is arranged in the line 6 between the reservoir 4 and the applying means 7. The metering means 10 may be a tap or an electrical controllable valve. It may also be arranged directly within the applying means 7. The metering means 10 is used for controlling the supply of photo-sensitizer 5 to the applying apertures 9 and, hence, onto the workpiece 2. For example, the metering means 10 may control the supply in such a way that photosensitizer 5 is applied onto the workpiece in regular temporal intervals.

Further, the device 1 comprises a laser 11 which generates pulsed laser radiation 12. In the best mode of the invention, the laser 11 is an ultra short pulse laser, in particular a femtosecond laser 11 with pulse durations in the range of several femtoseconds (fs) up to several 100 fs. For example, fiber oscillators may be used in order to reduce maintenance requirements. Due the shortness of the laser pulse, the laser radiation 12 has a comparably large spectrum. This spectrum is chosen or adjusted in such a way that the workpiece 2 is transparent at at least one central wavelength λ₀ of the laser 11, but preferably over the complete spectral range of the laser 11. The spectral range of the laser 11 is further chosen in such a way that it comprises radiation at twice the wavelength of an absorption peak of the photo-sensitizer 5, with a deviation of +/−10%. Depending on the employed photo-sensitizer 5, the spectral range of the laser 11 may therefore be, for example, in the range of 600 nm to 1200 nm, i.e. in the red or near infrared spectral region.

The laser 11 has a repetition rate of several megahertz (MHz), wherein the energy of one single laser pulse is in the range of picojoule (pJ) to nanojoule (nJ). In particular, the pulse energy should be variable. Since the invention may already be carried out at such low pulse energies, a subsequent amplification of the laser pulses is not necessary, although it may certainly be provided.

The radiation 12 of the laser 11 is guided to the workpiece 2 via a beam guiding system 13. The beam guiding system 13 comprises scanner means 14 with two pivotable scanner mirrors. Via the pivoting movement of the scanner mirrors, the laser beam 12 is laterally deflectable. Further, the beam guiding system 13 comprises a focusing optics 15, which is shown here schematically as a focusing lens. The focusing optics 15 concentrates the laser beam 12 onto a focus 16. Depending on the mutual positions of the focusing optics 15 and the workpiece 2, the focus 16 may be situated on the surface 8 of the workpiece 2 or—as shown in the drawing—in the bulk of the workpiece 2. The depth of the focus 16 in the workpiece 2, i.e the distance of the focus 16 from the surface 8, may be adapted or varied by a movement of the positioning means 3 and/or by shifting the focusing optics 15. The focusing optics 15 is controllable in its focusing properties, in particular with respect to the position of the focus and with respect to the focusing power, i.e. with respect to the size of the focal volume. Hence, the position of the focus 16 of the laser 11 may be three dimensionally varied by means of the focusing optics and the scanner means 14, in order to place the focus 16 at any desired position on or within the workpiece 2.

Finally, the device 1 also comprises a control 17, for example a programmable microprocessor. Via data lines 18 connecting the control 17 with the laser 11, with the scanner means 14, with the focusing optics 15, with the positioning means 3, and with the metering means 10, the control 17 may control all these elements, such that the device 1 may also be operated automatically. For example, the control may operate the elements 14, 15 of the beam guiding system 13 in such a way that the position of the focus 16 is only varied between two consecutive laser pulses. Further, the control 17 may suitably operate the metering means 10 for a controlled, regular application of photo-sensitizer 5 onto the workpiece 2.

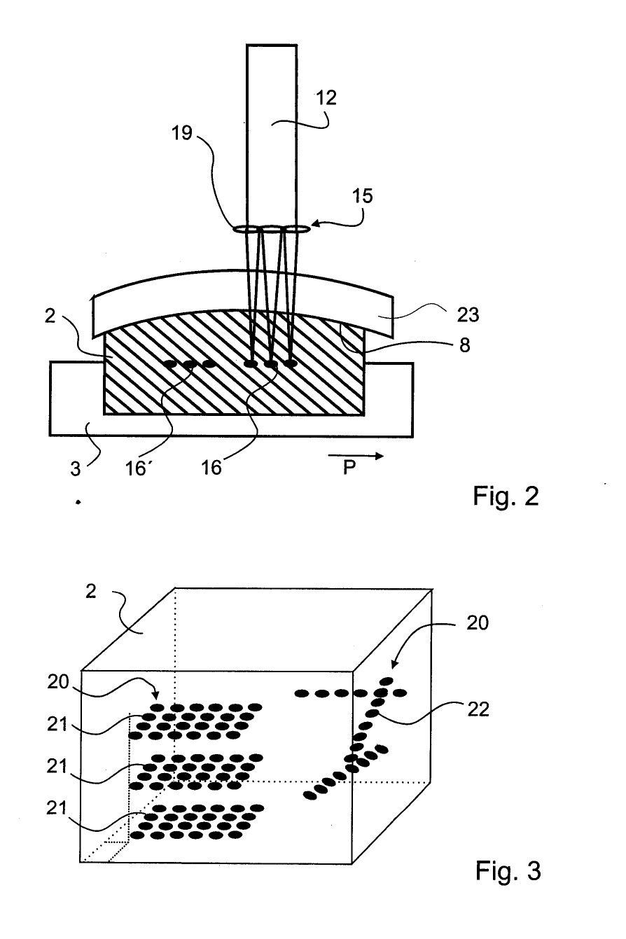

A portion of a variation of the device 1 is shown in FIG. 2. In contrast to the device 1 described previously, the focusing optics 15 now comprises a multi-focal optics, i.e. a focusing optics 15 simultaneously generating more than one focus 16. For this purpose, a lens array 19 may be employed, three of the lenses of which are shown here. Each lens generates its own focus 16, such that one single pulse of the laser radiation 12 results in three foci 16, and the workpiece may be processing simultaneously at three loci. In this way, the processing may be accelerated considerably.

If the positioning means 3 in FIG. 2 is moved into the direction designated with the arrow P, the next laser pulse generates three new foci 16′. These foci 16′are shown here spatially separated from the original foci 16. However, they may alternatively be located directly adjacent the original foci 16 or overlapped with these.

Another difference with respect to the device shown in FIG. 1 is the circumstance that in the embodiment of FIG. 2, a shaping member 23 is set onto the workpiece. By means of the shaping member 23, the surface 8 of the workpiece 2 is brought into a convex shape. In combination with the positioning means 3, the workpiece 2 is forced into a predetermined shape during the irradiation, and it is stably held in this shape. The shaping member 23, for example a hard contact lens, is transparent for the laser radiation 12, such that the radiation 12 may impinge on the workpiece 2 without attenuation.

The method according to the present invention may be performed with the device 1 by initially positioning the workpiece 2 in the positioning means 3 and fixing the workpiece 2, if necessary. Consecutively, a photo-sensitizer 5 is applied onto the workpiece 2 via the applying means 7, for example in the form of drops. The photo-sensitizer 5 is absorbed by the workpiece 2 and permeates also into deeper layers of the workpiece 2. After a certain waiting time in order to allow the photo-sensitizer 5 to soak the workpiece 2, irradiation of the workpiece 2 by the laser 11 is commenced. By focusing the short laser pulse with the intensity of the radiation 12 at the position of the focus 16 becomes so high that the photo-sensitizer 5 is activated there. Depending on the choice of chemical properties of the sensitizer, it may produce a cross linking or hardening of the workpiece 2 at the focus 16, such that the workpiece 2 becomes harder at this position.

During the irradiation, preferably between two consecutive laser pulses, the position of the focus 16 within the workpiece 2 may be varied by a variation of the focusing objects 15 and/or by moving the position means 3. In the course of the processing, the focus 16 of the laser 16 (or the simultaneously generated foci 16′) scan the complete irradiation zones 20.

Examples for such irradiation zones 20 are shown in FIG. 3. On the left side three planar radiation planes 21 are arranged one over the other, such that together they form a 3-dimensional irradiation zone 20. Each of the irradiation planes 21, in turn, is constituted by a plurality of irradiated focal areas. On the right hand side of workpiece 2 there are three line-shaped irradiation lines 22, which are also constituted of a plurality of irradiated focal points. The irradiation lines 22 intersect or overlap in pairs, such that together they form another irradiation zone 20. Non-irradiated or non-processed areas of the workpiece 2 are located between the lines 22. The irradiation lines 22 may be arranged in any desired way on or within the workpiece 2. For example, they may mutually intersect in such a way a web-like arrangement of irradiation lines 22 is generated.

By irradiating not only a single focal area 16 of the workpiece 2, but 1-, 2-, or 3-dimensional irradiation zones 20, the workpiece 2 is stabilized or hardened over wide areas, finally resulting in a stabilization of the complete workpiece 2.

Starting from the discussed embodiments, the invention may be varied in several ways. For example, in principle, any pulsed laser 11 may be used, with or without amplification of the pulses. It is also conceivable to use a laser that is adjustable in its spectrum in order to be able to adjust the laser 11 to a wavelength in which the activation of the photo-sensitizer 5 is particularly efficient. Further, a number of sensors may be provided in order to monitor the method, for example sensors for measuring the properties of the laser, of the metering means, or the position of the focus. It may also be considered adequate to connect such sensors with the control 17 in order to thereby influence the control of the processing method.

1. Method for processing an at least partially fluid-absorbent and in at least one spectral region transparent workpiece, wherein
the workpiece is irradiated with pulsed and focussed laser radiation,
the spectral range of the laser radiation comprises at least one wavelength in the transparent spectral region of the workpiece,
the focus of the laser radiation is situated on or within the workpiece,
before and/or during the irradiation a fluid photo-sensitizer is applied onto the workpiece and ingresses into the workpiece,
the photo-sensitizer comprises an absorption peak at or near half said at least one wavelength of the laser radiation and
the photo-sensitizer causes a hardening of the workpiece upon being irradiated.

2. Method according to claim 1, wherein the parameters of the laser radiation and of the focussing are chosen such that the activation of the photo-sensitizer is confined to the focal region of the radiation.

3. Method according to claim 1, wherein the position of the focus of the laser radiation on or within the workpiece is variable during the processing.

4. Method according to claim 1, wherein the focus of the laser radiation is guided in such a way that it scans one-dimensional irradiation zones on or within the workpiece.

5. Method according to claim 1, wherein the focus of the laser radiation is guided in such a way that it scans two-dimensional irradiation zones on or within the workpiece.

6. Method according to claim 1, wherein the focus of the laser radiation is guided in such a way that it scans three-dimensional irradiation zones within the workpiece.

7. Method according to claim 1, wherein the focus of the laser radiation is guided in such a way that it scans line-shaped irradiation zones, and at least two of these line-shaped irradiation zones intersect.

8. Method according to claim 1, wherein the laser radiation is focussed simultaneously onto more than one locus.

9. Method according to claim 1, wherein the laser radiation comprises radiation from the red or infrared region.

10. Method according to claim 1, wherein the laser is a short pulse or an ultrashort pulse laser.

11. Method according to claim 1, wherein the laser pulses are nanosecond pulses, picosecond pulses, femtosecond pulses or attosecond pulses.

12. Method according to claim 1, wherein the energy of one laser pulse is at or between 1 pJ (picojoule) and several 100 nJ (nanojoule).

13. Method according to claim 1, wherein the repetition rate of the laser is at or between 100 Hz and several 100 MHz.

14. Method according to claim 1, wherein the laser radiation is applied in such a way that on one locus on or within the workpiece an energy density of 0.1 kJ/cm² up to several 100 kJ/cm² is deposited.

15. Method according to claim 1, wherein the laser radiation is applied in such a way that on one locus on or within the workpiece an energy density of 10 kJ/cm² up to 200 kJ/cm² is deposited.

16. Method according to claim 1, wherein a photo-sensitizer with an absorption peak in the ultraviolet spectral region is used.

17. Method according to claim 1, wherein Riboflavin is used as a photo-sensitizer.

18. Method according to claim 1, wherein the workpiece is maintained in a predetermined shape by means of a shaping member during the processing.

19. Method according to claim 18, wherein the shaping member is transparent for the laser radiation, and the laser radiation is applied through the shaping member.

20. Calculation of the positions of a plurality of foci in preparation for performing a method according to claim 1.

21. Device for processing a workpiece, comprising a short pulse laser or ultrashort pulse laser, comprising a beam guiding system comprising a focussing element, and comprising a means for applying a photo-sensitizer onto the workpiece, in particular for performing a method according to claim 1.

22. Device according to claim 20, wherein the energy of one laser pulse is 1 pJ to several 100 nJ.

23. Device according to claim 20, wherein the laser is a nano-, pico-, femto-, or attosecond pulse laser.

24. Device according to claim 20, wherein the laser radiation comprises radiation from the red and/or infrared spectral region.

25. Device according to claim 20, further comprising a positioning means for positioning the workpiece.

26. Device according to claim 20, wherein the beam guiding system comprises controllable focusing optics, by means of which the position of the focus of the laser radiation is controllable.

27. Device according to claim 20, wherein the beam guiding system comprises scanning means.

28. Device according to claim 20, wherein the beam guiding system comprises focusing optics simultaneously generating more than one focus.

29. Device according to claim 20, wherein the average laser power is 0.5 mW to 1000 mW.

30. Device according to claim 20, further comprising a reservoir for the photo-sensitizer.

31. Device according to claim 20, wherein the means for applying the photo-sensitizer comprises metering means.

32. Device according to claim 20, further comprising a shaping member adapted to maintain the workpiece in a predetermined shape during the irradiation.

33. Device according to claim 20, further comprising a control for controlling the laser and the elements of the beam guiding system.

34. Device according to claim 20, further comprising a control for controlling the means for applying the photosensitizer.

35. Use of a short pulse laser of ultra short pulse laser for producing a device for treating keratoconus.

36. Method according to claim 1, wherein the workpiece is a cornea of an eye.
